(19) United States
(12) Patent Application Publication
(10) Pub. No.: US 2007/0106110 A1
(43) Pub. Date: May 10, 2007

(54) Sexercise ball

(76) Inventors: Rita G. Hollingsworth, Clinton, MD (US); Ronald A. Clark, Clinton, MD (US)

Correspondence Address: RONALD A. CLARK, 11402 ACCOLADE COURT, CLINTON, MD, 20735, US

(21) Appl. No.: 11/270,074

(22) Filed: Nov. 10, 2005

(51) Int. Cl. A61F 5/00

(52) U.S. Cl. 600/38

(57) ABSTRACT

A Sexual Stimulated Apparatus attached to A Rubber handle Exercise Ball that is mounted..

### FIELD OF INVENTION

The invention is related to a sexual apparatus.

### DESCRIPTION OF RELATED ART

A sexual apparatus is typically used for sexual stimulation.

### SUMMARY OF INVENTION

This invention combines sexual stimulation with exercise via rubber exercise ball which is mounted. It consist of a rubber handle and a rubber exercise ball with a adaptable sexual apparatus mount.

### BRIEF DESCRIPTION OF THE DRAWINGS

The invention is described in detail with regards to the following figures, in which like elements are

Referred to with like numerals an in which:

FIG. 1 is an exemplary diagram of a top view of the present invention.

FIG. 2 is an exemplary diagram of a side view of the present invention.

FIG. 3 is an exemplary diagram of an attachable dildo.

FIG. 4 is an exemplary diagram of the side view of additional embodiments.

### DETAILED DESCRIPTION OF DRAWINGS AND PREFERRED EMBODIMENTS

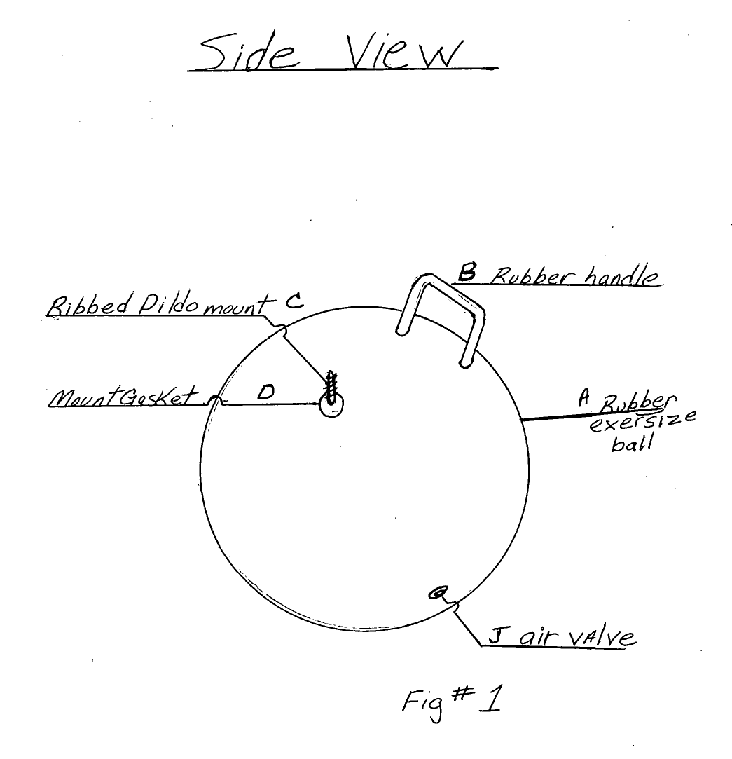

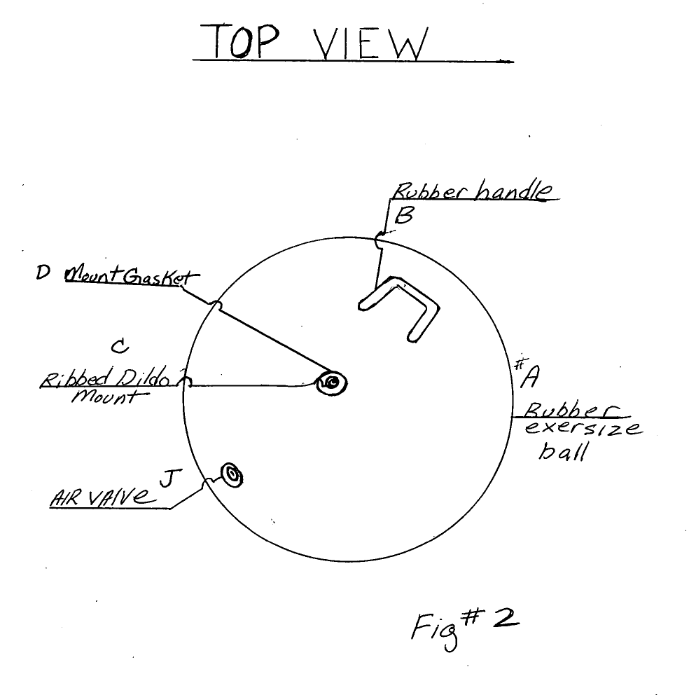

FIG. 1 A is a Rubber Exercise Ball

FIG. 1B is the Rubber Exercise Ball's Handle

FIG. 1C is the Plastic or Rubber Screw Ribbed Dildo Mount

FIG. 1D is the Base Gasket of the Dildo Mount which is attached to the Rubber Ball

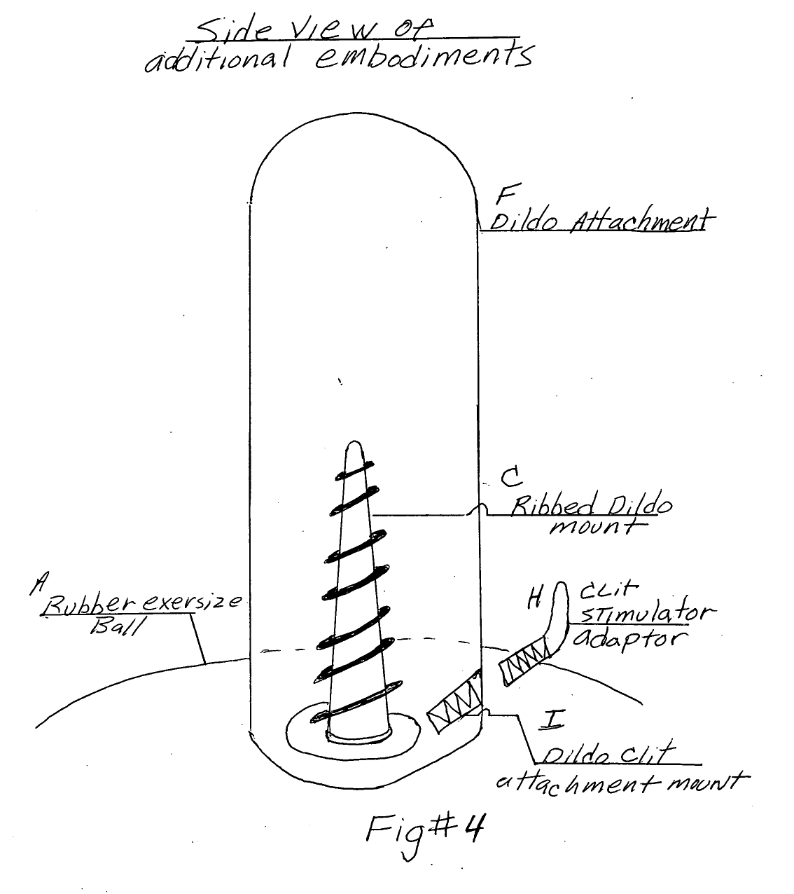

FIG. 1E is the Screw/Ribbed Dildo Mount which secure the Dildo F in FIG. 3. To C in FIG. 1.

FIG. 1 J is the Air Valve

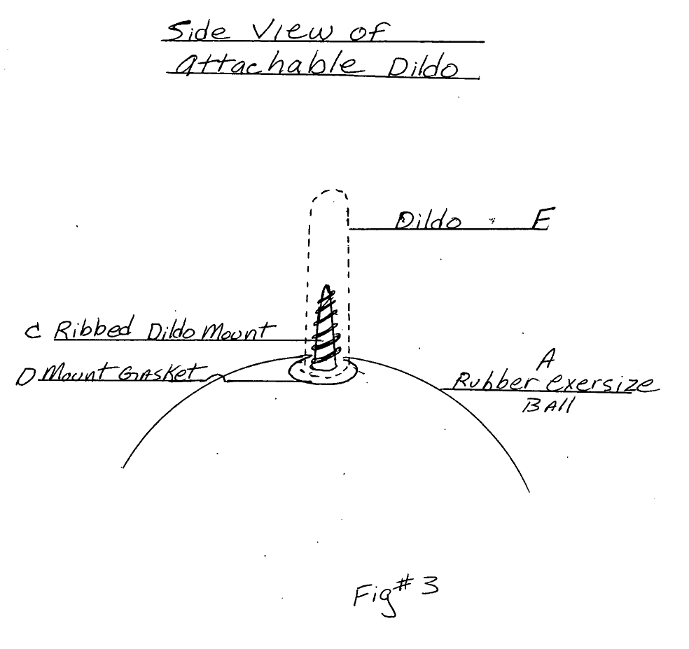

FIG. 2 Is an exemplary diagram top view FIG. 1A,B,C,D,E & J are the same as in FIG. 1.

FIG. 3 A is the Rubber Exercise Ball as shown in FIG. 1.

FIG. 3 C is the Dildo mount as shown in FIG. 1.

FIG. 3 D is the base gasket of the Dildo Mount C as shown in FIG. 1.

FIG. 3 F is the Dildo Apparatus attachment which secures to C in FIG. 1.

FIG. 3 G is the hollowed cavity opening to secure Dildo F. to Dildo Mount C in FIG. 1

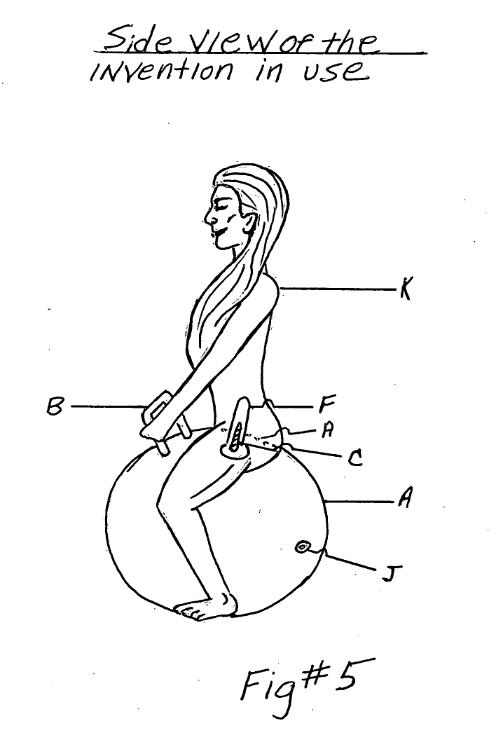

FIG. 4 is and exemplary diagram side view with additional embodiments C is the same as in FIG. 1, F is the Same as in FIG. 3, F also can come in various sizes as in FIG. 3.

FIG. 4 H is an attachment clit stimulator which attaches to F in FIG. 4 secured by FIG. 1 clit mount to F in FIG. 4

FIG. 5 is an exemplary diagram of the invention which a women is mounting Exercise Ball that has internal View shown with dotted lines showing apparatus C, D, & F.

FIG. 5 A, B, C, C, F & J are the same as in FIG. 1 & FIG. 3.

What is claimed:

1. I Claim that my Invention is for sexual stimulation and exercising.

2. I claim that this invention consist of a inflatable rubber exercise ball With rubber handle and attachable sexual apparatuses mounts.

3. I claim that the Rubber Exercise Ball will have multiple Female Apparatus Mounts.
